(19) United States
(12) Patent Application Publication
(10) Pub. No.: US 2017/0202259 A1
(43) Pub. Date: Jul. 20, 2017

(54) GLUCOSYLATED STEVIOL GLYCOSIDES AS A FLAVOR MODIFIER

(71) Applicant: PureCircle Sdn Bhd, Kuala Lumpur (MY)

(72) Inventor: Siddhartha PURKAYASTHA, Chicago, IL (US)

(73) Assignee: PureCircle Sdn Bhd, Kuala Lumpur (MY)

(21) Appl. No.: 15/478,808

(22) Filed: Apr. 4, 2017

Related U.S. Application Data
(63) Continuation of application No. 14/873,858, filed on Oct. 2, 2015.
Application No. 14/873,858 is a division of application No. 13/841,261, filed on Mar. 15, 2013, now abandoned.
Application No. 13/841,261 is a continuation-in-part of application No. PCT/US2012/030210, filed on Mar. 22, 2012.
Application No. PCT/US2012/030210 is a continuation-in-part of application No. PCT/US2011/033912, filed on Apr. 26, 2011.
(60) Provisional application No. 61/466,150, filed on Mar. 22, 2011.
(60) Provisional application No. 61/466,150, filed on Mar. 22, 2011.

(51) Int. Cl. A23L 27/30; A23L 2/02; A21D 2/18; C12P 19/56; A23C 9/123; A23L 7/126; A23L 2/60; A23L 19/00; A23L 27/00; A23C 9/156

(52) U.S. Cl. CPC A23L 27/36; A23L 27/33; A23L 27/88; A23L 2/02; A21D 2/181; A23C 9/156; A23C 9/123; A23L 7/126; A23L 2/60; A23L 19/00; C12P 19/56; A23V 2002/00

(57) ABSTRACT

A taste and flavor profile modifying composition is described. The composition includes glucosylated steviol glycosides which can modify the intensity of a taste and/or a flavor in a food or beverage product.

### RELATED APPLICATIONS

This application is a continuation-in-part application of and claims the benefit of priority to International PCT application PCT/US2012/030210, filed Mar. 22, 2012, which claims priority to International PCT application PCT/US2011/033912, filed Apr. 26, 2011; both of which claim the benefit of U.S. patent application Ser. No. 61/466,150, filed Mar. 22, 2011.

### BACKGROUND OF THE INVENTION

Field of the Invention

The present invention is directed to a taste and flavor profile modifying composition. The composition includes glucosylated steviol glycosides which can modified the intensity of a taste and/or a flavor in a food or beverage product.

Description of the Related Art

The extract of Stevia rebaudiana plant contains a mixture of different sweet diterpene glycosides, which have a single base—steviol and differ by the presence of carbohydrate residues at positions C13 and C19. These glycosides accumulate in Stevia leaves and compose approximately 10%-20% of the total dry weight. Typically, on a dry weight basis, the four major glycosides found in the leaves of Stevia are Dulcoside A (0.3%), Rebaudioside C (0.6%), Rebaudioside A (3.8%) and Stevioside (9.1%). Other glycosides identified in Stevia extract include Rebaudioside B, C, D, E, and F, Steviolbioside and Rubusoside.

The physical and sensory properties are well studied only for Stevioside and Rebaudioside A. They were tested for stability in carbonated beverages and found to be both heat and pH stable (Chang and Cook, 1983). The sweetness potency of Stevioside is around 210 times higher than sucrose, Rebaudioside A in between 200 and 400 times, and Rebaudioside C and Dulcoside A around 30 times (Phillips, 1989 and Tanaka, 1997).

However, apart from its high level of sweetness, they have also intrinsic properties of post-bitter taste and unpleasant and undesirable aftertaste. Some undesirable taste characteristics of glycosides can be as a result of contamination of other substances, presented in extract.

One of the main ways to improve the taste quality is the enzymatic glycosylation of mixture of semi-purified steviol glycosides. It is known that the undesired taste attributes can be substantially reduced or eliminated by the reaction of intermolecular transglycosylation of various enzymes, upon which the attachment of new carbohydrates at positions C13 and C19 of steviol glycosides takes place. Tanaka (1997) evaluated the effect of adding glucose molecules to purified stevioside molecules by tranglycosylation. The resulting glucosylated steviosides were evaluated for their sweetness and taste quality.

With an increase in the number of glucose units in steviol glycoside molecules (for example, from stevioside to Rebaudioside A), the sweetness intensity increases and sweetness profile (taste) improves. However, the relative sweetness does not increase significantly beyond a certain level with a further increase of glucose units, as shown in FIG. 1a. The published data show that the sweetness quality improves with the addition of glucose units, but does not explicitly or implicitly mention that the addition of glucose units contributes to a reduction of sweetness.

### SUMMARY OF INVENTION

The present invention is directed to a taste and flavor profile improving composition. The composition includes glucosylated steviol glycosides which can modified the intensity of a taste and/or a flavor in a food or beverage product. In some embodiments, the glucosylated steviol glycosides may include a plurality of glucose units. For example, the glucosylated steviol glycosides may include three, four, five, or more than five glucose units.

The present invention is also directed to a food or beverage product having an intense taste and flavor profile, wherein the food or beverage product includes a taste and flavor modifying composition comprising glucosylated steviol glycosides. A wide range of food and beverage products, such as, but not limited to, carbonated soft drinks, fruit juices, dairy foods, dairy beverages, baked goods, cereal products, and table top sweeteners, may be made in accordance with the present invention. The taste and flavor profile of a food or beverage product including a taste and flavor modifying composition, wherein the taste and flavor modifying composition includes glucosylated steviol glycosides, may be more intense than a comparative taste and flavor profile of a comparative food or beverage product which does not include the taste and flavor modifying composition. Moreover, the mouthfeel of a food or beverage product including the taste and flavor modifying composition, wherein the taste and flavor modifying composition includes glucosylated steviol glycosides, may be improved in relation to a mouthfeel of a comparative food or beverage product which does not include the taste and flavor modifying composition.

The present invention is further directed to a method of enhancing the taste and flavor intensity of a food or beverage product, including the step of adding a taste and flavor modifying composition to the food or beverage product, wherein the taste and flavor modifying composition includes glucosylated steviol glycosides. The present invention is also directed to a method of improving the organoleptic properties of a food or beverage product including a high fructose syrup, including the step of adding the taste and flavor modifying composition to the food or beverage product. For example, adding the taste and flavor modifying composition may cause the high fructose syrup, such as high fructose corn syrup, to taste more like sugar. Also, if the high fructose syrup is high fructose corn syrup 42 (HFCS 42), adding the taste and flavor modifying composition may cause the HFCS 42 to taste more like high fructose corn syrup 55 (HFCS 55).

The present invention is also directed to a method of making a taste and flavor modifying composition, including: extracting steviol glycosides from leaves of a Steviol rebaudiana Bertoni plant, and transglycosylating the steviol glycosides to add glucose units to the steviol glycosides. In some embodiments, transglycosylating the steviol glycosides includes enzymatic transglycosylation using an enzyme. Examples of enzymes which may be used in accordance with the present invention include, but are not limited to, pullulanase, isomaltase, β-galactosidase, dextrine saccharase, and cyclodextrin glucotransferase.

The present invention is further directed to a method of making a food or beverage product, including: adding a taste and flavor modifying composition including glucosylated steviol glycosides, and adding a reduced amount of erythritol, wherein the reduced amount of erythritol is less than the amount of erythritol in a comparative food or beverage composition which does not include the taste and flavor modifying composition. The mouthfeel of the food or beverage product is similar to the mouthfeel of the comparative food or beverage product, even though the comparative food or beverage product contains a higher level of erythritol.

The foregoing has outlined rather broadly the features and technical advantages of the present invention in order that the detailed description of the invention that follows may be better understood. Additional features of the invention which form the subject of the claims of the invention will be described hereinafter. It should be appreciated by those skilled in the art that the specific embodiments disclosed may be readily utilized as a basis for modifying or designing other methods or structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description.

### BRIEF DESCRIPTION OF THE DRAWINGS

FIG. 1a is a bar graph showing the relative sweetness of steviol glycoside molecules with increasing numbers of glucose units. FIG. 1b is a bar graph showing the taste quality of steviol glycoside molecules with increasing numbers of glucose units.

FIG. 2 shows the chemical structures of stevioside and glucosyl stevioside.

FIG. 3 is a graph of the sweetness potency, or sucrose equivalent value (SEV), of stevia glycosides at a 5% sugar sweetness level. FIG. 3A is a graph of an average sweet perception ranking of aqueous samples with sucrose and glucosylated steviol glycosides. FIG. 3B is a graph of the effect of glucosylated steviol glycoside concentration on sweetness detection in carbonated soft drinks.

FIG. 4a is a graph of the relative ratings of the sweetness intensity and sweet onset of mango-passion fruit flavored water beverages to which different stevia ingredients were added, and which did not include sugar. FIG. 4b is a graph of the relative ratings of the passion fruit flavor and mango flavor of these mango-passion fruit flavored water beverages.

FIG. 5a is a graph of the relative ratings of the sweetness intensity and sweet onset of mango-passion fruit flavored water beverages to which different stevia ingredients were added, and which included sugar. FIG. 5b is a graph of the relative ratings of the passion fruit flavor and mango flavor of these mango-passion fruit flavored water beverages.

FIG. 6 is a graph of the SEV (sucrose equivalent value) contribution of NSF-02 plotted against the concentration of NSF-02, in acidified water and in acidified sugar solutions.

FIG. 7 is a graph showing the synergy of steviol glycoside with GSG, plotted against the concentration of GSG.

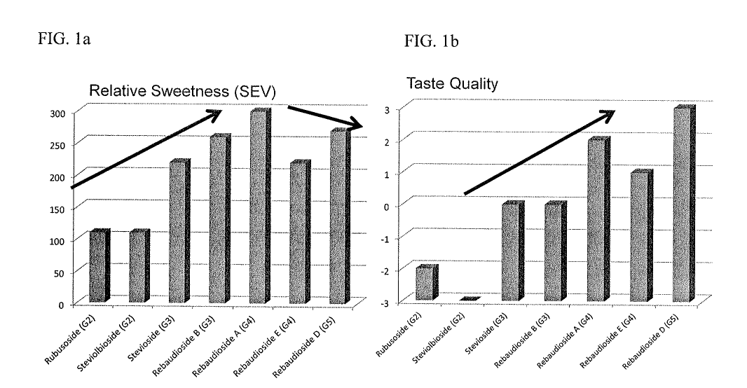

### DETAILED DESCRIPTION

It has been unexpectedly discovered that glucosylation of steviol glycosides beyond a certain number of glucose units effectively reduces sweetness. It has also been discovered that with the reduction of sweetness, the glucosylated steviol glycosides can contribute to the modification of flavor and sweetness profiles. Therefore, while sweetness decreases with glucosylation, flavor modification increases. The steviol glycosides mixture provides a certain amount of sweetness, but the present invention shows that the glucosylated steviol glycosides (hereinafter “GSG”) modified the flavor and sweetness profile in a wide range of applications, such as those listed in, but not limited by, the categories shown below in Table 2.

### Table 2: GSG Applications

TABLE 2
GSG Applications

| Application | Product | Results |
|---|---|---|
| Beverages | Apple Blueberry | Significantly more berry |
| | Juice Drink | flavor intensity than the |
| | | Control. Directionally, more |
| | | acceptable for overall flavor. |
| | Orange Pineapple | Significantly more orange |
| | Passion Fruit | flavor intensity, pineapple |
| | Carbonated Drink | flavor intensity, and |
| | | sweetness intensity. |
| Baked | Lemon Poppy | Significantly more overall |
| Goods | Seed Muffins | flavor intensity and sweetness |
| | | intensity than the Control. |
| | | Directionally, the Test |
| | | sample was more acceptable |
| | | overall and had more lemon |
| | | flavor intensity |
| Processed | Strawberry Topping | Significantly less bitter |
| Fruits | | than the control. |
| Dairy | Banana Flavored | Significantly more overall |
| | Milk Drink | flavor intensity, banana flavor |
| | | intensity, sweetness intensity, |
| | | tartness intensity and |
| | | bitterness intensity. |

Similar taste and flavor improvements were found in other categories of products, including, but not limited to, table top sweeteners, sauces and gravies, confectionery products, baked goods, cereals, snacks, and fruit and vegetable preparations.

### Examples

In the following examples, the percentages in the formulas refer to percentages by weight.

### Example 1: Evaluation of Iso-Sweetness of Steviol Glycosides

To evaluate the iso-sweetness of steviol glycosides (SG) and glucosylated steviol glycosides (GSG), a series of samples were selected as shown below in Table 3. The GSG was produced by treating the raw materials, steviol glycosides extracted from the Stevia plant, and starch extracted from tapioca, with a natural enzyme. The enzyme transfers glucose units from starch to the steviol glycosides. The enzyme used to facilitate this transfer is produced by means of fermentation using non-GMO (non-genetically modified organism) bacteria.

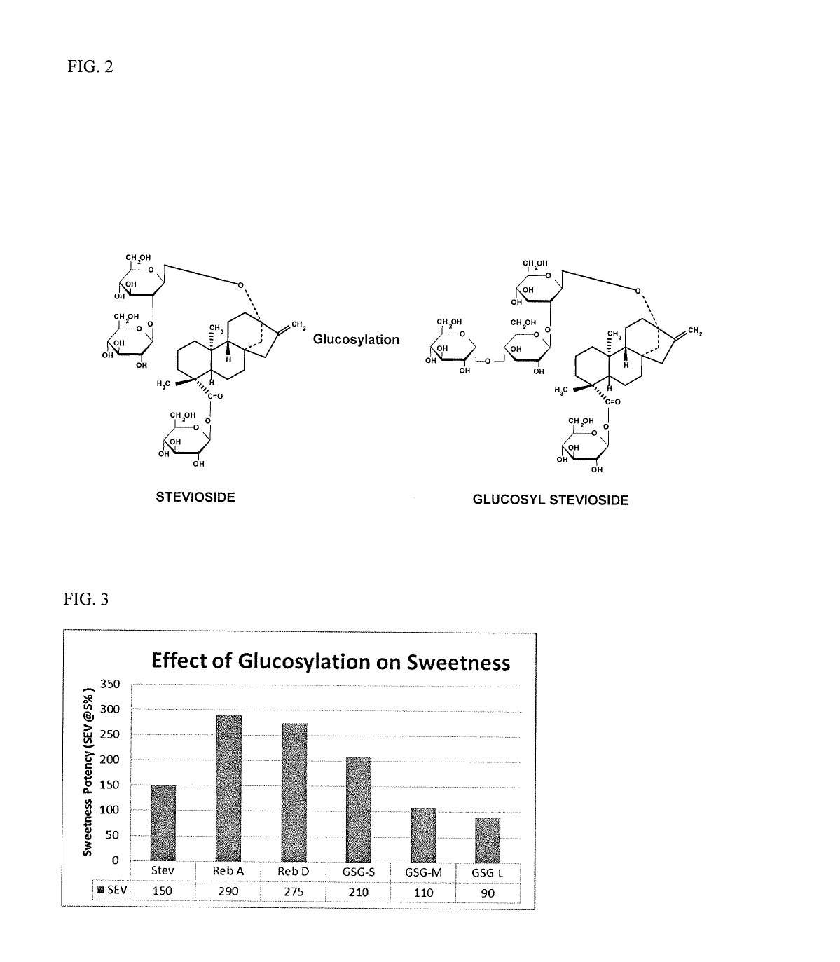

FIG. 2 is an illustration of an example of glucosylation. Specifically, FIG. 2 illustrates the single glucosylation (G1) of a stevioside molecule. This process can yield multiple glucosylation (G2, G3, etc.) of different steviol glycosides (mainly stevioside and Rebaudioside A) present in stevia extract.

TABLE 3

| | | | Non- | Glucosylated Steviol | | |
|---|---|---|---|---|---|---|
| | Molec- | | glucosylated | Glycosides | | |
| Test | ular | Steviol | Steviol | G1 & | | G10- |
| Ingredients | Wt (avg) | equivalent | Glycosides | G2 | G3-G9 | G20 |
| Stevioside | 805 | 0.396 | >99% | | | |
| Reb A | 967 | 0.329 | >99% | | | |
| Reb D | 1129 | 0.282 | >99% | | | |
| GSG-S | 1210 | 0.263 | <10% | 95% | | |
| GSG-M | 1380 | 0.231 | 7.80% | 42% | 50% | |
| GSG-L | 1798 | 0.177 | 5% | 19% | 60% | 16% |

To evaluate the sweetness potency of various concentrations of stevia products in aqueous solutions, aqueous solutions of sugar, stevioside, Rebaudioside A (Reb A), Rebaudioside D (Reb D), GSG-S (contains mainly smaller GSGs with 1 or 2 glucose units added to SG), GSG-M and GSG-L at various concentrations were prepared using bottled water. Samples were evaluated by the judges at room temperature (70-72° F.).

The judges were 11 panelists that have been previously qualified for their taste acuity and trained in the use of a sweetness intensity rating scale. The evaluations were done in duplicate using the same panelists so that a total of 22 values were generated for each average data point. Prior to the conduct of the study, judges were trained with sugar solutions and the use of the ballot.

Samples were given to the judges sequentially and coded with triple digit numbers. The order of sample presentation was randomized to avoid order of presentation bias. A rest period of five minutes was provided between samples. Water and unsalted crackers were provided in order to cleanse the palate.

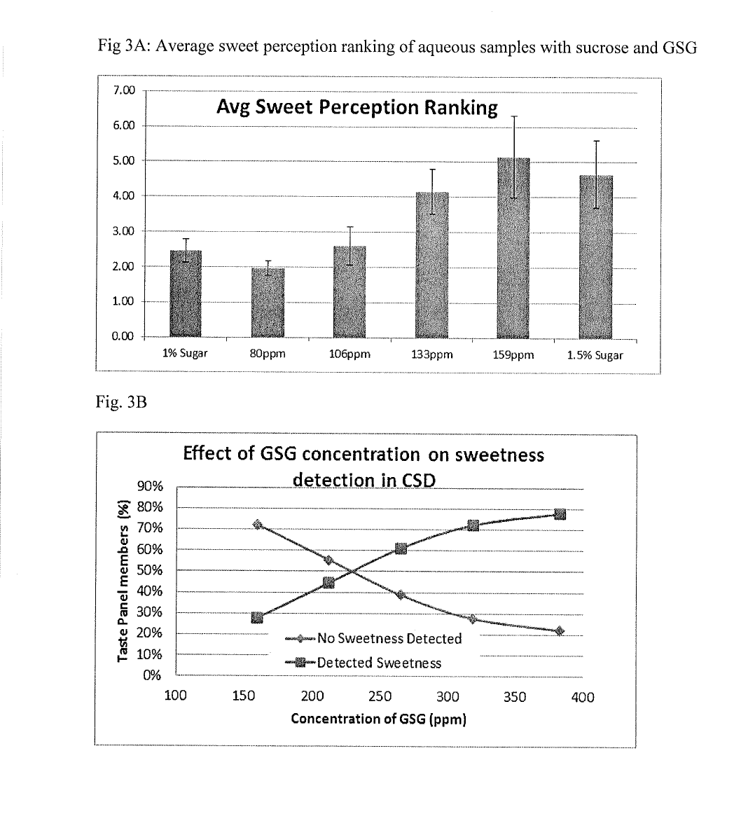

Results were statistically analyzed to generate a standard error value for each solution as well as a confidence level at a 95% level. By comparing the sweetness of each test ingredient to the sweetness of several sucrose solutions, the sweetness potency of different stevia ingredients was estimated as shown in FIG. 3. FIG. 3 is a graph of the sweetness potency, or sucrose equivalent value (SEV), of different stevia ingredients at a 5% sugar sweetness level (i.e. at a concentration equivalent to 5% sucrose). This figure shows the effect of glucosylation on the SEV of steviol glycosides. As the number of glucose units on steviol glycosides increases, the sweetness increases from stevioside to Reb A and then starts decreasing with additional glucose units.

### Example 1A: Detection of Concentration Threshold for Sweetness Detection

Thirty-three consumer panel members evaluated a series of aqueous solutions of sucrose and GSG at room temperature; the sucrose solutions of 1.0% & 1.5% concentration and GSG solutions with concentrations ranging between 80 and 160 ppm were prepared with filter water. The objective of the test was to determine the sweetness detection limit of GSG.

The samples were coded and presented in random order to panel members to taste and rank them from “least sweet” (rank 1) to the “most sweet” (rank 6). Panelists were asked to focus only on sweet attribute of those samples and to use cracker and water in order to cleanse the palate between samples.

TABLE 3A
Methodology for Sensory Evaluation

| | |
|---|---|
| Nature of Participants: | Company employees |
| Number of Sessions | 2 |
| Number of Participants: | 33 |
| Test Design: | Randomized samples. Blind |
| Sensory Test Method: | Perception of sweetness rating |
| Environmental Condition | Standard room lighting |
| Attributes and Scales: | |

Forced ranking samples according to perception of sweetness where
1 = Least sweet Perception 6 = Most sweet perception

| | |
|---|---|
| Statistical Analysis: | ANOVA (by Block) |
| Sample Size | ~2.0 oz. in a clear capped 3.5 oz. plastic |
| | drinking cup |
| Serving Temperature | Room temperature (~20° C.) |
| Serving/Panelists | Samples served simultaneously. Panelists |
| Instruction: | were instructed to taste and rank them from |
| | least to most |

The ranking of all samples by 33 consumer panel members was analyzed to determine the average and standard deviation of the numerical ranking of each sample containing either sucrose or GSG; results are graphically presented in FIG. 3A. The overall sweetness of those samples was barely detectable. The ranking shows that 80 ppm GSG solution was the least sweet sample and the sample with 159 ppm GSG ranked as the sweetest samples. The sweetness detection of GSG in water was at a concentration higher than 106 ppm and below 133 ppm, thus the detection limit is considered at 125 ppm

### Example 1B: Sweetness Detection of Concentration Threshold for Sweetness Detection

Eighteen consumer panel members participated in a sensory test to detect sweetness in five lemon-lime Carbonated Soft Drink (CSD) samples sweetened with different amount of GSG ranging between 150 and 400 ppm. The percentage of panel members who detected the sweetness for each sample was calculated and plotted as shown in FIG. 3B. More than 50% panel members could detect sweetness in samples containing more than 250 ppm of GSG.

The CSD samples were made with 50 g syrup (Table 3B), GSG solution and required amount of carbonated water to make 300 ml beverage for different level of GSG concentration. The GSG concentration in the five test samples were 159, 212, 265, 318 and 383 ppm.

TABLE 3B
Syrup formulation for CSD

| | |
|---|---|
| Water | |
| Citric Acid | 2.2 |
| Lemon Lime Flavor | 1.6 |
| Sodium Benzoate | 0.4 |
| Potassium Citrate | 0.4 |
| Xanthan Gum | 0.1 |
| Total (wt.) | |

All five test samples were coded and presented to panel members in random order to detect sweetness. Crackers and water were provided in order to cleanse the palate between each sample. The panel members were asked to indicate in which samples they could detect sweetness, the choice of response was Yes or No.

TABLE 3C
Methodology for Sensory Evaluation

| | |
|---|---|
| Nature of Participants: | Company employees |
| Number of Session | 1 |
| Number of Participants: | 18 (55% Male, 45% Female) |
| Test Design: | Randomized samples. Blind |
| Sensory Test Method: | Sweet perception-Forced choice |
| Environmental Condition | Standard room lighting |
| Attributes and Scales: | Detection of Sweetness; Forced choice- |
| | Yes/No |
| Sample Size | ~2.0 oz. in a clear capped 3.5 oz. |
| | plastic drinking cup |
| Serving Temperature | Room temperature (~20° C.) |
| Serving/Panelists | Samples served simultaneously. Panelists |
| Instruction: | were instructed to taste and note whether |
| | they can detect any sweetness. |

The majority of panel members (56%) could not detect sweetness at 212 ppm of GSG (NSF-02, available from Pure Circle) and 39% panel members could not detect sweetness at 265 ppm GSG. FIG. 3B shows the GSG concentration vs the percentage of the panel members who could (or not) detect sweetness in the carbonated soft drink sample sweetened with GSG only. Since, more than 50% panel members could detect sweetness in samples containing more than 250 ppm of GSG, the threshold concentration of GSG that imparts sweetness perception is 250 ppm in a typical Carbonated Soft Drink beverage.

### Example 2: Effect of Glucosylation on Flavor Modification

A mango-passion fruit flavored water formula was developed to evaluate the effect of different stevia ingredients on the sweetness and flavor profile of the beverage. A total of 9-10 panel members participated in this sensory test, where they assigned relative values to sweetness, onset of sweetness, mango fruit flavor, passion fruit flavor, acidity, overall taste, etc.

### Example 2A: Effect of Glucosylation on Flavor Modification of No-Sugar-Added Beverage

Table 4 shows the no-sugar added beverage formula that used mainly Reb A, Reb D, GSG-S, or GSG-L. The amount of each ingredient (Reb A: 150 ppm; Reb D: 165 ppm; GSG-S: 190 ppm; and GSG-L: 300 ppm) was selected to have around 50 ppm of steviol in each formula.

TABLE 4
Mango-Passion Fruit Flavored Beverage
(No Sugar Added)

| Stevia Ingredient | Reb A | Reb D | GSG-S | GSG-L |
|---|---|---|---|---|
| Water | 95.82 | 95.82 | 95.82 | 95.82 |
| SG Content | 0.015 | 0.0165 | 0.019 | 0.03 |
| Citric Acid | 0.078 | 0.078 | 0.078 | 0.078 |
| Sodium Citrate | 0.056 | 0.056 | 0.056 | 0.056 |
| Mango flavor | 0.031 | 0.031 | 0.031 | 0.031 |
| Passion fruit flavor | 0.014 | 0.014 | 0.014 | 0.014 |

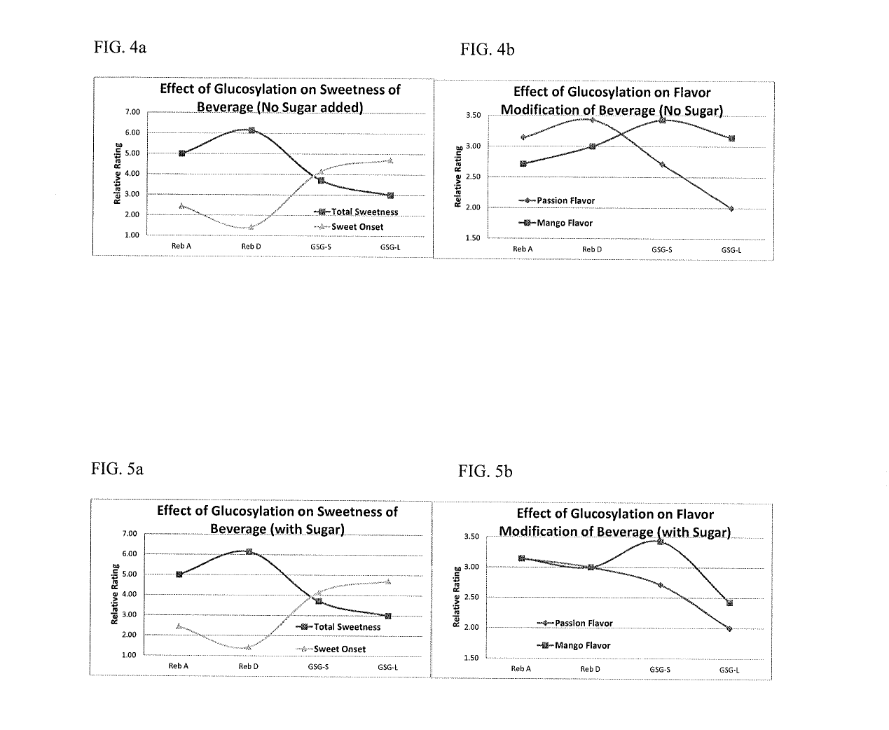

FIGS. 4a-4b show the modification of flavor and sweetness profiles caused by glucosylation. The sweetness intensity decreased and sweet onset delayed with glucosylation. Mango flavor was modified and the passion fruit flavor reduced with glucosylation.

### Example 2B: Effect of Glucosylation on Flavor Modification of a Beverage with Sugar

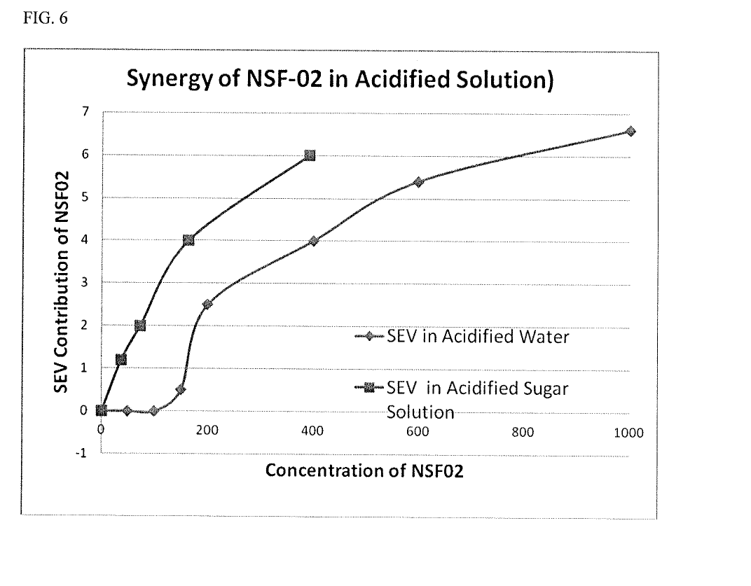

Table 5 shows the same mango-passion fruit flavored beverage formula with 4% sugar and stevia ingredients that contribute an additional 4% sugar-equivalent sweetness. The formula used Reb A, Reb D, GSG-S, or GSG-L in the amount of 50, 55, 73, and 200 ppm respectively as outlined in the formula below. As the number of glucose units increased more flavor improvement (whether modification or suppression) was observed as shown in FIGS. 5a-5b.

TABLE 5
Mango-Passion Fruit Flavored Beverage (Sugar Added)

| | Reb A | Reb D | GSG-S | GSG-L |
|---|---|---|---|---|
| Stevia Ingredient | (%) | (%) | (%) | (%) |
| Water | 95.82 | 95.82 | 95.82 | 95.82 |
| Sugar | 4.00 | 4.00 | 4.00 | 4.00 |
| SG Content | 0.005 | 0.0055 | 0.0073 | 0.02 |
| Citric Acid | 0.078 | 0.078 | 0.078 | 0.078 |
| Sodium Citrate | 0.056 | 0.056 | 0.056 | 0.056 |
| Mango flavor | 0.031 | 0.031 | 0.031 | 0.031 |
| Passion fruit flavor | 0.014 | 0.014 | 0.014 | 0.014 |

FIGS. 5a-5b show the effect of glucosylation of SG on the modification of flavor and sweetness profiles of a beverage sweetened with sugar and stevia (SG). The sweetness intensity decreased and sweet onset delayed with glucosylation. Both Mango and passion fruit flavors were somewhat suppressed with glucosylation. A key point is that GSG modified the flavor profile.

### Example 3: Juice Drink with Glucosyl Steviol Glycosides

In this and the following examples, the GSG may be any glucosyl steviol glycoside composition, such as, but not limited to, a combination of GSG-S, GSG-M, and GSG-L.

To evaluate the flavor modification in a juice drink, a range of GSG concentrations (0 to 1000 ppm) was used with a typical apple blueberry juice drink formula. The objective was to assess whether the addition of GSG has an effect on key flavor attributes in various beverage applications. Specifically, the objective was to determine whether the flavor profile and overall acceptance of a Control sample of apple blueberry juice (containing no GSG) differs from a 30% reduced sugar Test sample of the same beverage (containing GSG). After preliminary sensory tests, it was apparent that GSG modified the flavor and sweetness profile at all concentrations. The GSG concentration is preferably in the range of about 0 to 1000 ppm, more preferably in the range of about 1 to 500 ppm, and most preferably in the range of about 1 to 250 ppm.

For detailed sensory tests, two samples were selected to test against the control sample. The methodology of the detailed sensory tests is set forth below in Table 6. The formulas of the control and test samples are set forth below in Table 7.

TABLE 6
Methodology

| | |
|---|---|
| Number of Sessions | 1 |
| Number of Participants: | 20 |
| Test Design: | Balanced, randomized within pair. Blind |
| Sensory Test Method: | Intensity and acceptance ratings |
| Environmental Condition | Standard booth lighting |
| Attributes and Scales: | |

Overall Acceptance on a 6-pt hedonic scale where 5 = Like Extremely,
2 = Neither Like Nor Dislike, and 0 = Dislike Extremely
Overall Flavor, Apple Flavor, Berry Flavor, Sweetness, Tartness, Bitterness
and Lingering Sweet Aftertaste Intensity on a 6-pt continuous intensity
scale where 0 = Imperceptible and 5 = Extremely Pronounced
Open Ended General Comments

| | |
|---|---|
| Serving Temperature | Refrigerated temperature (~45° F.) |
| Serving/Panelists | Samples served simultaneously. Panelists |
| Instruction: | instructed to read ingredient statement, |
| | evaluate each sample. |

TABLE 7
Samples

| | | Test-1 | Test-2 |
|---|---|---|---|
| | | Low | Hi |
| Description Flavor | Control | GSG Level | GSG Level |
| Apple Juice Concentrate | 6.21% | 6.21% | 6.21% |
| Blueberry Juice Concentrate | 0.76% | 0.76% | 0.76% |
| Apple and blueberry flavor | 0.12% | 0.12% | 0.12% |
| Sugar | 5.60% | 5.60% | 5.60% |
| GSG | | 0.025% | 0.05% |
| Water | Balance | Balance | Balance |

In this study, twenty consumer panel members evaluated three samples of apple blueberry flavored juice drink for overall acceptance and attribute intensities of apple and berry flavors, onset of flavor, sweetness, and aftertaste (includes tartness, bitterness and lingering sweet aftertaste intensity). The three samples included a full sugar control sample containing no Glucosyl Steviol Glycosides (GSG) and two test samples containing low (0.025%) and high (0.05%) levels of GSG. The objective of the test was to determine if the addition of Glucosyl Steviol Glycosides affects the flavor profile of a juice drink. The results indicated:
The Test samples had significantly higher overall acceptability and more apple flavor intensity than the Control sample (at >90% confidence).
The sweetness intensity of the Test sample with low GSG was not significantly different from the control. GSG modifieds sweetness and flavor profile at high level of usage (p=0.047).
There was no significant difference in aftertaste intensities between the test and control samples (at 90% confidence).
   It was clearly evident that GSG modified the flavor and sweetness profile at all concentrations. The GSG concentration is preferably in the range of about 0 to 1000 ppm, more preferably in the range of about 1 to 500 ppm, and most preferably in the range of about 1 to 250 ppm.

### Example 4: Flavored Carbonated Soft Drink with Glucosyl Steviol Glycosides

To evaluate the flavor modification in a carbonated soft drink, a range of GSG concentrations (0 to 1000 ppm) was used with an orange-pineapple flavored carbonated soft drink formula. The objective was to assess whether the addition of GSG has an effect on key flavor attributes in various carbonated soft drink (CSD) beverage applications. Specifically, the objective was to determine if the flavor profile and overall acceptance of a Control sample of orange pineapple carbonated drink differs from Test samples of the same beverage containing GSG. After preliminary sensory tests, it was apparent that GSG modified the flavor and sweetness profile at all concentrations. The GSG concentration is preferably in the range of about 0 to 1000 ppm, more preferably in the range of about 0 to 500 ppm, and most preferably in the range of about 1 to 250 ppm.

For detailed sensory tests, two samples were selected to test against the control sample. The methodology of the detailed sensory tests is set forth below in Table 8. The formulas of the control and test samples are set forth below in Table 9.

TABLE 8
Methodology

| | |
|---|---|
| Number of Sessions | 1 |
| Number of Participants: | 24 |
| Test Design: | Balanced, randomized within pair. Blind. |
| Sensory Test Method: | Intensity and acceptance ratings |
| Environmental Condition | Standard booth lighting |
| Attributes and Scales: | |

Overall Acceptance on a 6-pt hedonic scale where 5 = Like and
0 = Dislike
Overall Flavor, Orange Flavor, Pineapple Flavor, Aftertaste,
and Sweetness 6-pt continuous intensity scale where 0 =
Imperceptible and 5 = Extremely Pronounced

| | |
|---|---|
| Statistical Analysis: | ANOVA (by Block) with Post Hoc |
| | Duncan's Test |
| Sample Size | ~1.5 oz. in a clear capped plastic cup |
| Serving Temperature | Refrigerated temperature (~45° F.) |
| Serving/Panelists | Samples served simultaneously. Panelists |
| Instruction: | instructed to read ingredient statement, |
| | evaluate each sample. |

TABLE 9
Samples

| | Control | Test 1 | Test 2 |
|---|---|---|---|
| Ingredients | (%) | (%) | (%) |
| Water | 90.8 | 90.8 | 90.8 |
| Sugar | 8.93 | 8.93 | 8.93 |
| Acids | 0.142 | 0.142 | 0.142 |
| Orange Flavor | 0.069 | 0.069 | 0.069 |
| Pineapple Flavor | 0.050 | 0.050 | 0.050 |
| SG95 | 0.006 | 0.006 | 0.006 |
| GSG | | 0.025 | 0.05 |

In this study, twenty-four members consumer panel evaluated three samples of orange pineapple fruit flavored carbonated drink for overall acceptance and attribute intensities (overall flavor, orange flavor, pineapple flavor, sweetness, and aftertaste). The three samples included: a reduced sugar control sample containing SG95 (a stevia extract) and two test samples that were the same as the Control sample plus GSG added at 0.025% (low) and 0.05% (High) levels. SG95 is a high purity stevia sweetener available from PureCircle, 915 Harger Road, Suite 250, Oak Brook, Ill. 60523, USA. The objective of the test was to determine if the addition of stevia extract solids affects the flavor profile of a reduced sugar carbonated drink. The results indicated:
The Test sample with low GSG had significantly more orange flavor intensity than the Control sample (95% confidence).

The Test sample with high GSG also displayed more orange flavor intensity than the Control sample (p=0.089).
There was no significant difference in overall acceptance, pineapple flavor intensity, or aftertaste intensity between the control and two test samples (at 90% confidence).

It was clearly evident that GSG modified the flavor and sweetness profile at both low (250 ppm) and high (500 ppm) concentrations. The GSG concentration for flavor and taste profile modification is preferably in the range of about 0 to 1000 ppm, more preferably in the range of about 1 to 500 ppm, and most preferably in the range of about 1 to 250 ppm.

### Example 5: Flavored Milk Drink with Glucosyl Steviol Glycosides

To evaluate the flavor modification in a flavored dairy beverage, a range of GSG concentrations (0 to 1000 ppm) was used with a banana flavored beverage formula. The objective was to determine if the addition of Glucosyl Steviol Glycosides (GSG) has an effect on key flavor attributes and/or improves flavor perception in various beverage applications, specifically in dairy beverages. Specifically, the objective was to determine if the flavor profile and overall acceptance of a Control sample of banana flavored milk drink (containing no GSG) differs from two Test samples of the same drink containing two levels of GSG. Though the test was conducted with banana flavor, the findings are also pertinent with all fruit, vegetable, chocolate, coco flavored beverages and energy drinks. After preliminary sensory tests, it was apparent that GSG modified the flavor and sweetness profile at all concentrations.

For detailed sensory tests, two samples were selected to test against the control sample. The methodology of the detailed sensory tests is set forth below in Table 10. The formulas of the control and test samples are set forth below in Table 11.

TABLE 10
Methodology

| | |
|---|---|
| Number of Sessions | 1 |
| Number of Participants: | 32 |
| Test Design: | Balanced, randomized within pair. Blind. |
| Sensory Test Method: | Intensity and acceptance ratings |
| Environmental Condition | Standard booth lighting |
| Attributes and Scales: | |

Overall Acceptance on a 9-pt hedonic scale where 9 = Like Extremely,
5 = Neither Like Nor Dislike, and 1 = Dislike Extremely
Overall Flavor, Banana Flavor, Dairy Flavor, Sweetness, Tartness and
Bitterness Intensity on a 11-pt continuous intensity scale where 0 =
Imperceptible and 10 = Extremely Pronounced

| | |
|---|---|
| Statistical Analysis: | ANOVA (by Block) with Post Hoc |
| | Duncan's Test |
| Sample Size | ~2.0 oz. in a clear capped 3.5 oz. plastic |
| | drinking cup |
| Serving Temperature | Refrigerated temperature (~45° F.) |
| Serving/Panelists | Samples served simultaneously. Panelists |
| Instruction: | instructed to read ingredient statement, |
| | evaluate each sample. |

TABLE 11
Samples
Control: Sugar and Stevia Extract (Reb A)
Test: Sugar, Stevia Extract (Reb A) & GSG

| | | Test-1 | Test-2 |
|---|---|---|---|
| | | Low | High |
| Ingredients | Control | GSG Level | GSG Level |
| MILK | 95.6% | 95.6 % | 95.6% |
| (2% fat) | | | |
| SUGAR | 4.2 | 4.2 | 4.2 |
| BANANA | 0.20 | 0.20 | 0.20 |
| FLV | | | |
| Reb A | 0.006 | 0.006 | 0.006 |
| GSG | | 0.0175 | 0.035 |
| TOTAL | 100 | 100 | 100 |

In this study, thirty-two consumer panel members evaluated three samples of reduced sugar banana flavored milk drink for overall acceptance and attribute intensities (overall flavor, banana flavor, dairy flavor, sweetness, and aftertaste intensity). The three samples included: a control sample sweetened with sugar and stevia extract (Reb A) and containing no Glucosyl Steviol Glycosides (GSG) and two test samples sweetened with sugar and stevia extract (Reb A) containing Glucosyl Steviol Glycosides (GSG). One of the test samples contained a low (0.0175%) amount of GSG and the other one contained a high (0.035%) amount of GSG. The objective of the test was to determine if the addition of Glucosyl Steviol Glycosides affects/improves the flavor profile of a banana flavored milk drink. The results indicated:
The Test sample containing a high level of GSG had significantly more banana flavor intensity, sweetness intensity, and delayed onset of dairy flavor note than the Control sample (at 95% confidence).
The test sample containing a low level of GSG also contributed to higher banana flavor intensity (at >90% confidence).
There was no significant difference in overall acceptance, dairy flavor intensity, onset of banana flavor and aftertaste between the test samples and control.

The GSG concentration in flavored milk and other dairy products is preferably in the range of about 0 to 750 ppm, more preferably in the range of about 1 to 500 ppm, and most preferably in the range of about 1 to 350 ppm.

### Example 6: Baked Goods with Glucosyl Steviol Glycosides

To evaluate the flavor modification in baked goods, a range of GSG concentration (0 to 5000 ppm) was used with a lemon poppy seed muffin formula. The objective was to determine if the addition of Glucosyl Steviol Glycosides has an effect on key flavor attributes and/or improves flavor perception in various food applications, specifically in various baked goods. Specifically, the objective was to determine if the flavor profile and overall acceptance of a Control sample of lemon poppy seed muffin (containing no Glucosyl Steviol Glycosides) differs from a Test sample of the same muffin (containing Glucosyl Steviol Glycosides). Though the test was conducted with muffins, the findings are also pertinent with all baked goods, not limited to cookies, cakes, pastries, bread, etc. After preliminary sensory tests, it was apparent that GSG modified the flavor and sweetness profile at all concentrations. The GSG concentration for baked goods is preferably in the range of about 0 to 1500 ppm.

For detailed sensory tests, two samples were selected to test against the control sample. The methodology of the detailed sensory tests is set forth below in Table 12. The formulas of the control and test samples are set forth below in Table 13.

TABLE 12
Methodology

| | |
|---|---|
| Number of Sessions | 1 |
| Number of Participants: | 35 |
| Test Design: | Balanced, randomized within pair. Blind. |
| Sensory Test Method: | Intensity and acceptance ratings |
| Environmental Condition | Standard booth lighting |
| Attributes and Scales: | |

Overall Acceptance on a 9-pt hedonic scale where 9 = Like Extremely,
5 = Neither Like Nor Dislike, and 1 = Dislike Extremely
Overall Flavor, Lemon Flavor, Sweetness, Tartness and Bitterness
Intensity on a 11-pt continuous intensity scale where 0 = Imperceptible
and 10 = Extremely Pronounced
Open Ended General Comments
Gender and Age

| | |
|---|---|
| Statistical Analysis: | ANOVA (by Block) with Post Hoc |
| | Duncan's Test |
| Sample Size | ¼ muffin (~14 grams) in a 3.5 oz. |
| | plastic soufflé cup |
| Serving Temperature | ambient temperature (~68° F.) |
| Serving/Panelists | Samples served simultaneously. Panelists |
| Instruction: | instructed to read ingredient statement, |
| | evaluate each sample. |

TABLE 13
Samples

| | Control | Test |
|---|---|---|
| Ingredients | (%) | (%) |
| AP Flour | 24.37 | 24.35 |
| Milk | 24.01 | 23.99 |
| Sugar | 23.16 | 23.14 |
| Vegetable Oil | 14.79 | 14.78 |
| Eggs | 9.54 | 9.53 |
| Poppy Seeds | 1.00 | 1.00 |
| Lemon Flavor | 0.83 | 0.83 |
| Salt | 0.72 | 0.71 |
| Lemon Juice | 0.59 | 0.59 |
| Vanilla Extract | 0.52 | 0.52 |
| Baking Powder | 0.48 | 0.48 |
| GSG | | 0.07 |
| TOTAL | 100.00 | 100.00 |

In this study, thirty-five consumer panel members evaluated two samples of lemon poppy seed muffins for overall acceptance and attribute intensities (overall flavor, lemon flavor, sweetness, tartness and bitterness intensity). The two samples included: 1) a Control sample containing no Glucosyl Steviol Glycosides (GSG) and 2) a Test sample containing GSG. The objective of the test was to determine if the addition of Glucosyl Steviol Glycosides affects/improves the flavor profile of a lemon poppy seed muffin. The results indicated:
The Test sample (containing Glucosyl Steviol Glycosides) had significantly more overall flavor intensity and sweetness intensity than the Control (at 90% confidence).
There was no significant difference in overall flavor acceptance, lemon flavor intensity, tartness intensity or bitterness intensity between the two samples (at 90% confidence). Directionally, the Test sample was more acceptable overall and had more lemon flavor intensity than the Control (p values=0.124 and 0.190 respectively).
Based on panelist comments, the Control sample had less lemon flavor than the Test sample.

The GSG concentration is preferably in the range of about 0 to 1500 ppm, more preferably in the range of about 0 to 750 ppm, and most preferably in the range of about 1 to 400 ppm.

### Example 7: Spreads & Fruit/Vegetable Preparations with Glucosyl Steviol Glycosides

To evaluate the flavor modification in fruit/vegetable spreads and fruit preparations, a range of GSG concentration (0 to 5000 ppm) was used with a strawberry topping formula for spread. The objective was to determine if the addition of Glucosyl Steviol Glycosides has an effect on key flavor attributes and/or improves flavor perception in various food applications, specifically in fruit or vegetable preparations. Specifically, the objective was to determine if the flavor profile and overall acceptance of a Control sample of strawberry topping (containing no GSG) differs from a Test sample of the same topping (containing GSG). Though the test was conducted with strawberry spread/fruit prep/topping, the findings are also pertinent with fruit and vegetable preparations, not limited to fruits (banana, all berries, mango, etc.) and vegetables (celery, artichoke, squash, avocado, etc). After preliminary sensory tests, it was apparent that GSG modified the flavor and sweetness profile at all concentrations. The GSG concentration is preferably in the range of about 0 to 1000 ppm.

For detailed sensory tests, two samples were selected to test against the control sample. The methodology of the detailed sensory tests is set forth below in Table 14. The formulas of the control and test samples are set forth below in Table 15.

TABLE 14
Methodology

| | |
|---|---|
| Number of Sessions | 1 |
| Number of Participants: | 28 |
| Test Design: | Balanced, randomized within pair. Blind. |
| Sensory Test Method: | Intensity and acceptance ratings |
| Environmental Condition | Standard booth lighting |
| Attributes and Scales: | |

Overall Acceptance on a 9-pt hedonic scale where 9 = Like Extremely,
5 = Neither Like Nor Dislike, and 1 = Dislike Extremely
Overall Flavor, Fresh Strawberry Flavor, Sweetness, Tartness and
Bitterness Intensity on a 11-pt continuous intensity scale where 0 =
Imperceptible and 10 = Extremely Pronounced
Open Ended General Comments
Gender and Age

| | |
|---|---|
| Statistical Analysis: | ANOVA (by Block) with Post Hoc |
| | Duncan's Test |
| Sample Size | ~0.5 oz. in a clear capped 1 oz. plastic cup |
| Serving Temperature | Refrigerated temperature (~45° F.) |
| Serving/Panelists | Samples served simultaneously. |
| Instruction: | Panelists instructed to read ingredient |
| | statement, evaluate each sample. |

TABLE 15
Samples

| | Control | Test |
|---|---|---|
| Ingredients | (%) | (%) |
| Sugar | 39.23 | 39.18 |
| Water | 29.43 | 29.39 |
| Diced Strawberries | 15.44 | 15.42 |
| Strawberry Juice | 15.44 | 15.42 |
| Pectin | 0.26 | 0.26 |
| Potassium Benzoate | 0.10 | 0.10 |
| RebA | 0.06 | 0.06 |
| Citric Acid | 0.05 | 0.05 |
| GSG | | 0.022 |
| TOTAL | 100.00 | 100.00 |

In this study, twenty-eight consumer panel members evaluated two samples of strawberry flavored reduced sugar topping for overall acceptance and attribute intensities (overall flavor, fresh strawberry flavor, sweetness, tartness and bitterness intensity). The two samples included: 1) a control sample sweetened with sugar and Rebaudioside A containing no Glucosyl Steviol Glycosides (GSG) and 2) a test sample sweetened with sugar and Rebaudioside A containing GSG. The objective of the test was to determine if the addition of GSG affects/improves the flavor profile of strawberry flavored topping. The results indicated:
There was no significant difference in overall flavor acceptance, overall flavor intensity, fresh strawberry flavor intensity, sweetness intensity or tartness intensity (at 90% confidence).
The test sample was significantly less bitter than the control (at 90% confidence).
   The GSG concentration in fruit and vegetable spreads and preparations is preferably in the range of about 0 to 1000 ppm, more preferably in the range of about 1 to 600 ppm, and most preferably in the range of about 1 to 400 ppm.

### Example 8: Yogurt with GSG

To evaluate the flavor modification in flavored and unflavored yogurt, a range of GSG concentration (0 to 1000 ppm) was used with a vanilla flavored yogurt bought from a local store. Though the test was conducted with vanilla flavored yogurt, the findings are also pertinent with all other unflavored and flavored fermented dairy products like cheese, yogurt (with fat or no-fat), drinkable yogurts, smoothies, yogurt with fruit preparations not limited to fruit (banana, all berries, mango, etc.). After preliminary sensory tests, it was apparent that GSG modified the flavor and sweetness profile at all concentrations. The GSG concentration is preferably in the range of about 0 to 1000 ppm, more preferably in the range of about 50 to 500 ppm, and most preferably in the range of about 100 to 400 ppm.

Three vanilla-flavored yogurt samples (generic brand) were sweetened with sugar, sugar+Reb A and Sugar+Reb A+GSG. The amount of GSG was 220 ppm. The twelve member panel found that GSG modified the sweetness profile and positively impacted the flavor profile. GSG helped in rounding the sweetness profile and directionally helped in reducing the aftertaste of Reb A.

### Example 9: Lemon-Lime Carbonated Soft Drink with GSG

To evaluate the flavor modification in a flavored carbonated soft drink (CSD), a range of GSG concentration (0 to 1000 ppm) was used with a lemon-lime flavored CSD. Though the test was conducted with reduced sugar lemon-lime flavored CSD, the findings are also pertinent with all other flavored CSDs (cola, orange, grape fruit, passion fruit, berries fruit group, mango, etc.) with all levels of sugar and diet (no sugar) products. After preliminary sensory tests, it was apparent that GSG modifies the flavor and sweetness profile at all concentrations. The GSG concentration is preferably in the range of about 0 to 1000 ppm, more preferably in the range of about 0 to 500 ppm, and most preferably in the range of about 1 to 300 ppm.

Three CSD samples were made with sugar, Sugar+Reb A, and Sugar+Reb A+GSG. The amount of GSG was 310 ppm. The sample with GSG had directionally improved sweetness profile, modified lemon flavor note and reduced bitterness and aftertaste compared to the sample sweetened with sugar and Reb A only.

### Example 10. Chocolate Milk with GSG

To evaluate the flavor modification in flavored dairy beverages, a range of GSG concentration (0 to 1000 ppm) was used with chocolate milk formulations sweetened with sugar and/or high fructose corn syrup (HFCS) and stevia. Though the test was conducted with a reduced sugar chocolate flavored dairy beverage, the findings are also pertinent with all other flavored dairy beverages with different levels of fat or no-fat with different flavors (strawberry, blueberry, mango, etc.) with different levels of sugar including diet (no sugar) products. After preliminary sensory tests, it was apparent that GSG modifies the flavor and sweetness profile at all concentrations. The GSG concentration is preferably in the range of about 0 to 500 ppm, more preferably in the range of about 1 to 400 ppm, and most preferably in the range of about 1 to 300 ppm.

One of the test samples was sweetened with a mixture of HFCS42, sugar and Reb A; the other test sample also included 337 ppm of GSG. The GSG modified the chocolate flavor, dairy note and sweetness profile.

### Example 11: Baked Good Frosting with GSG

To evaluate the flavor modification in baked good frostings and spreads, a range of GSG concentration (0 to 0.5%) was used with vanilla flavored cake frosting formulations sweetened with sugar and/or high fructose corn syrup (HFCS) and stevia. Though the test was conducted with a reduced sugar frosting, the findings are also pertinent with all other flavored frostings with different levels of fat or no-fat and different flavors (chocolate, strawberry, blueberry, mango, etc.) with different levels of sugar including no-sugar-added products. After preliminary sensory tests, it was apparent that GSG modified the flavor and sweetness profile at all concentrations. The GSG concentration is preferably in the range of about 0 to 0.5%, more preferably in the range of about 0.1 to 0.4%, and most preferably in the range of about 0.2 to 0.3%.

As an example, a typical sensory test was conducted where GSG was added to a reduced sugar cake frosting. The amount of GSG was 0.23%, which modified the sweetness profile as more sugar-like as well as promoted the vanilla flavor.

### Example 12: Snacks with GSG

To evaluate the flavor modification in snacks and cereal/nut products, a range of GSG concentration (0 to 0.5%) was used with cinnamon flavored coated almonds sweetened with sugar and/or high fructose corn syrup (HFCS). Though the test was conducted with full sugar coated nuts, the findings are also pertinent with all other coatings used for snacks, cereal, confectionery with different level of moisture and fat (or no-fat) and different flavors (chocolate, cinnamon, hazelnut, maple, brown-sugar, strawberry, blueberry, mango, etc.) with different levels of sugar including no-sugar-added products. After preliminary sensory tests, it was apparent that GSG modified the flavor and sweetness profile at all concentrations. The GSG concentration is preferably in the range of about 0 to 0.5%.

As an example, two coated almond snacks were prepared where the test sample had reduced sugar. GSG was added in the amount of 0.19% to the test sample. Additional test showed that much lower amount of GSG (100 to 500 ppm) can contribute to the flavor profile and taste modification if any other sweetener is used with GSG. The GSG provided the rounded sweetness and modified cinnamon flavor to the test sample.

The GSG concentration is preferably in the range of about 0 to 0.5%, more preferably in the range of about 0.1 to 0.3%, and most preferably in the range of about 1 to 400 ppm.

### Example 13: Sweetness Synergy of GSG with Sugar

To evaluate the sweetness detection of GSG, a series of samples was made with NSF-02 in acidified water (pH=3.3, pH was adjusted using citric acid). NSF-02 contains GSG and about 15% to 20% dextrin, and is available from PureCircle, 915 Harger Road, Suite 250, Oak Brook, Ill. 60523, USA.

Aqueous solutions of GSG at various concentrations were prepared using bottled water that was acidified to a pH of about 3.5. The pH was adjusted using 1% citric acid solution within a narrow range. The concentrations of GSG ranged between 0 to 1000 ppm. Samples were evaluated by the judges at room temperature (70-72° F.).

The judges were 10 panelists that have been previously qualified for their taste acuity and trained in the use of a sweetness intensity rating scale. The evaluations were done in duplicate using the same panelists (N=20). Prior to the conduct of the study, judges were presented with sugar controls prepared with the acidified water for the intensity rating on the ballot referencing 2, 4, 6 and 8 on the evaluation scale. These solutions were provided to the judges in order to refresh the judge's memory with the intensity ratings.

Samples were given to the judges sequentially and coded with triple digit numbers. The order of sample presentation was randomized to avoid order of presentation bias. A rest period of five minutes was provided between samples. Water and unsalted crackers were provided in order to cleanse the palate. The judges could not detect any sweetness below 150 ppm of NSF-02.

If a small amount of GSG is used with a sweetened beverage (caloric or non-caloric), the product is perceived sweeter. However, the same amount may not contribute any sweetness perception in an unsweetened product. The flavor modification can be perceived also at the small concentration.

To quantify the synergy between GSG (NSF-02) and sugar, acidified flavored water beverage (˜3.5 pH) samples were prepared with combination of different level of sugar and GSG to have 8% sucrose-equivalent-sweetness. The amount of GSG needed to reduce sugar by 1%, 2%, 4% and 6% were estimated by a trained 5 panel members and plotted in FIG. 6 (upper line). The iso-sweet data (SEV or sugar equivalent sweetness value) were generated for GSG in acidified water and plotted in FIG. 6 (lower line).

Note that GSG did not contribute any detectable level of sweetness at low levels of GSG (0 to 150 ppm). The synergy between sugar and GSG is the difference in SEV contribution of GSG with and without sugar. At a concentration greater than 1 ppm, GSG not only modifies flavor, but it can also increase sweetness perception in the presence of other sweeteners. The synergistic effect between the NSF-02 and other caloric and non-caloric sweetener is observed at a concentration of GSG preferably greater than about 1 ppm and distinctly observed up to the concentration of 250 ppm. At a higher concentration between 500 ppm and above in acidified solution, the sweetness is more additive (of GSG and other sweetener) than the synergistic effect that can be found in a lower concentration.

Accordingly, the present invention shows that GSG not only modifies flavor, but it can also modify sweetness profile and modify sweetness perception in the presence of other sweeteners. This modification of sweetness is caused by the synergy between GSG and other sweeteners.

### Example 14: GSG Synergy with HFCS

The objective of this experiment was to develop the same sweetness and mouthfeel of High Fructose Corn Syrup (HFCS) 55 in water solution using an equal amount of HFCS 42 plus GSG. HFCS 55 contains a total of 77% dry solid, and 55% of the dry solid is fructose. HFCS 42 contains 71% dry solid, and 42% of the dry solid is fructose. The sweetness equivalence values (SEVs) of HFCS 55 and HFCS 42 are 0.99 and 0.91, respectively.

To match a similar sweetness profile and mouthfeel of HFCS 55, samples of HFCS 42 were tested including different amounts of GSG from 0 to 500 ppm and different biogums (Xanthan, Gum Arabic, CMC, guar, Locust bean gum, pectin), or polysaccharides (maltodextrin, oligosaccharides, resistant maltodextrin), or polyols. All combinations of GSG and bulking agent (to aid mouthfeel) provided the desired sweetness and mouthfeel that matched HFCS 55. The GSG concentration is preferably in the range of about 0 to 500 ppm, more preferably in the range of about 1 to 300 ppm, and most preferably in the range of about 1 ppm to 250 ppm. However, the best solution was the combination of Xanthan gum and GSG (shown in Table 16). A similar analysis was carried out in a lemon-lime CSD application, which had a combination of GSG and Xanthan gum to provide the similar sweetness and mouthfeel with HFCS 42 as found in the formulation with HFCS 55.

TABLE 16
Sweetness profile modification of HFCS with GSG

| Ingredients | HFCS 55 | HFCS 42 |
|---|---|---|
| Water | 87.02 | 87.00 |
| HFCS 55 | 12.98 | — |
| HFCS 42 | — | 12.98 |
| Xanthan Gum | — | 0.0085 |
| GSG | — | 0.0068 |
| Brix | 10 | 9.30 |
| Total | 100 g | 100 g |

It was noticed that when Xanthan gum was added together with NSF-02 the mouth feel perception was improved as well as the overall flavor profile.
The addition of NSF-02 made the sample taste more like a sugar-based product.

Sensory analysis (discrimination test) was conducted to determine the difference between the beverage samples with HFCS 55 and HFCS 42. A triangle test was conducted with 19 panelists. Only 4 identified the correct sample, two of whom were guessing. The two that correctly identified the odd sample indicated the differences were due to the HFCS 42 being less acidic and more flavorful; they both also mentioned that HFCS 55 was slightly less sweet than HFCS 42.

Although the tests were conducted using high fructose corn syrups, the results are not limited to high fructose syrups made from corn. The results are also applicable to high fructose syrups made from other carbohydrate sources, such as, but not limited to, wheat, barley, tapioca, rice, and potatoes.

### Example 15: GSG Synergy with Other Non-Caloric Sweeteners

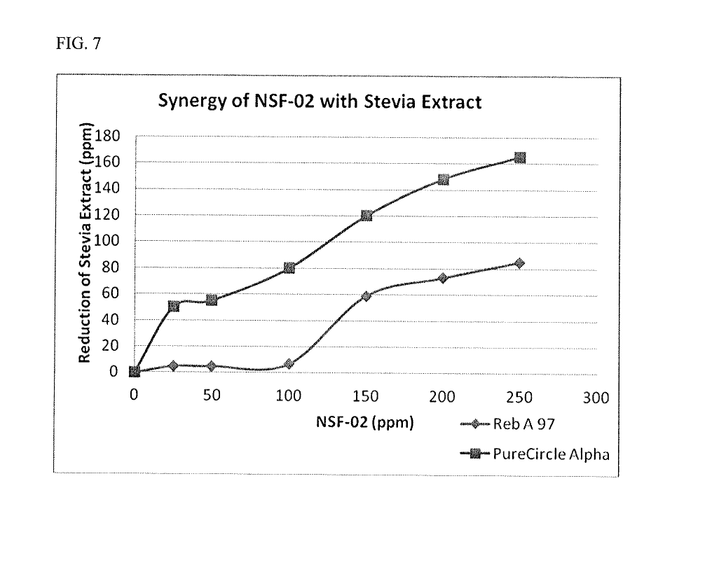

GSG was tested with several natural (Reb A, SG95 and PureCircle Alpha derived from stevia extract) and synthetic sweeteners (Sucralose, Acesulfame-K, cyclamate and aspartame) to investigate the synergy between GSG and high intensity sweeteners. While GSG modifies the flavor profile, it also shows a different degree of synergy with high intensity sweeteners. As an example, to estimate the synergy between GSG and stevia sweeteners, NSF-02 (GSG+dextrin) was mixed with a required quantity of PureCircle Alpha (a blend of selected steviol glycosides available from PureCircle) or Reb A 97 (PureCircle product) in acidic solution (pH=3.8) to attain 8% sugar equivalent sweetness as shown in the Table 17. Synergy was calculated as the reduction of stevia sweeteners (Alpha or Reb A) for addition of different level of NSF-02 as shown FIG. 7.

TABLE 17
Synergy of GSG with High-Intensity Sweetener

| | Control | ppm | ppm | ppm | ppm | ppm | ppm |
|---|---|---|---|---|---|---|---|
| NSF02 | 0 | 25 | 50 | 100 | 150 | 200 | 250 |
| Reb A | 345 | 340 | 340 | 338 | 286 | 272 | 260 |
| Alpha | 400 | 350 | 345 | 320 | 280 | 252 | 235 |
| Synergy with | 0 | 5 | 5 | 7 | 59 | 73 | 85 |
| Reb A | | | | | | | |
| Synergy with | 0 | 50 | 55 | 80 | 120 | 148 | 165 |
| Alpha | | | | | | | |

It was discovered that Alpha shows modified sweetness in the presence of a very small amount of NSF-02 (25 ppm or less), whereas more than 100 ppm NSF-02 had to be added to attain any synergy with Reb A as shown in FIG. 7. Note that the detection level of sweetness of GSG is around 150 ppm as shown in FIG. 6, In a solution with Alpha, the NSF-02 concentration is preferably greater than about 5 ppm, more preferably greater than about 25 ppm, and most preferably greater than about 100 ppm. In a solution with Reb A, the NSF-02 concentration is preferably greater than about 100 ppm, more preferably greater than about 150 ppm, and most preferably greater than about 200 ppm.

Although the tests were conducted using stevia-based sweeteners, the results are not limited to stevia extracts. The results are also applicable to other low or no calorie sweeteners, such as, but not limited to, sucralose, aspartame, thaumatin, NHDC, saccharine, acesulphame-k, mogrosides, monatin, neotame, altame, brazein, etc.

### Example 16: GSG as a Mouthfeel Modifier

GSG works with sugar, HFCS and other natural sweeteners to provide a better mouthfeel and sweetness profile in beverages. Erythritol is used with stevia in beverages to provide some sweetness, but mainly to contribute mouthfeel that is lacking when a high amount of sugar is replaced with high intensity sweetener. A study was conducted to investigate the amount of erythritol that can be replaced with GSG in still beverages.

Sample Preparation:

A number of acidified beverage samples were targeted to 8 Brix sweetness level with 200 ppm of Reb A and the combination of erythritol and GSG as shown in Table 17.

TABLE 18
Formulation to determine the contribution of GSG on mouthfeel

| | Erythritol Conc (%) | | | | |
|---|---|---|---|---|---|
| | 3.5 | 2.5 | 1.5 | 3 | 2.75 |
| Ingredient/Test ID | E | E6 | E7 | E8 | E9 |
| Reb A | 0.1 | 0.1 | 0.1 | 0.1 | 0.1 |
| GSG | | 0.044 | 0.0875 | 0.022 | 0.033 |
| Erythritol | 17.5 | 12.5 | 7.5 | 15 | 13.75 |
| Citric | 0.25 | 0.25 | 0.25 | 0.25 | 0.25 |
| Water | 482 | 487 | 492 | 484 | 486 |
| Total | 500 | 500 | 500 | 500 | 500 |

In a comparison of E, E6 and E7 samples, E6 was found to be very close to control (E) on flavor and overall mouthfeel, and slightly less sweet than control. E7 was very watery.

A triangle test with E and E6 samples was conducted over two days with a total of 12 panel members. Five of them detected the correct sample. The conclusion was that panel members could detect the difference; thus the test failed.

A preliminary test with E, E8, E9 was then conducted, a triangle test with E and E9 was run. Three out of ten panel members could identify the correct sample.

GSG can reduce erythritol usage by 20-30% (from 3.5% to 2.75-3%) in beverages without any sacrifice of taste or mouthfeel. In the flavor system studied, the GSG concentration is preferably greater than about 10 ppm, more preferably greater than about 20 ppm, and most preferably in the range of about 30 ppm to about 200 ppm. In some flavor systems, GSG may replace more erythritol to provide a balanced, rounded sweetness flavor.

Although the tests were conducted using erythritol, the results are not limited to erythritol as a reduced calorie bulking agent. The results are also applicable to other low or no calorie bulking agents, such as, but not limited to, maltitol, sorbitol, xylitol, maltulose, tagatose, resistant maltodextrin and oligosaccharides, hydrogenated oligosaccharides, etc.

### Example 17: GSG as a Flavor Modifier for Alcoholic Beverages

Steviol glycoside is soluble in both water and alcohol. The solubility and flavor/taste modification of GSG can be effectively used in different alcoholic beverages, including but not limited to beer, wine, spirits made from different source of carbohydrates as well as premade cocktails alcohol, such as margarita mix, mojito, screw driver, long island ice tea and beverages with moderate alcohol contents such as wine cooler, cider beverages, tea/lemonade malt beverages. Several products were tested with GSG and determined that GSG contributes to modification of the astringency, mouthfeel and overall flavor profile of alcoholic beverages. The GSG concentration in alcoholic beverages is preferably in the range of about 0 to 500 ppm, more preferably in the range of about 0 to 250 ppm.

### Example 18: GSG as a Flavor Modifier for Solid and Semi-Solid Foods

GSG can be used as flavor and taste modifier in the solid food (e.g., chewing gum, snacks, breakfast cereal, etc) and semi-solid food (e.g., paste, yogurt, fruit prep). The consumption of a dry foods needs increased level of mastication, with typical residence times in the mouth in the range of 20-30 seconds, depending on size, composition and texture of the food, and the physiology of the consumer. The beverage generally has a much shorter residence time in the mouth, in the range of few seconds at most. The solid foods, therefore, incorporate significant amounts of saliva in the mouth causing a dilution which can exceed 2 folds dilution in many cases. This dilution effect requires a higher use level of GSG in solid foods such as breakfast cereals, bakery, snacks, etc. compared to their beverage counterparts.

Furthermore, in for some solid foods, the “normal” method of consumption involves an additional pseudo-dilution. An example is breakfast cereals where a typical serving size of 30-40 g is typically consumed with the addition of 100-200 ml of milk leading to a dilution of 2 to 5 folds on average.

Different concentrations of GSG, ranging between 0 to 2000 ppm were tested as taste and favor modifier with different types food and semi-solid foods, such as sauce, gravy, salad dressings, breakfast cereals, condiment and relishes, confectionery, frostings, frozen dairy, snacks, soft candy, etc. The most effective use level that contributed flavor and taste modification in different food products are listed in the following table.

TABLE 19
Most effective use level of GSG in
different solid/semisolid food categories

| | Most Effective |
|---|---|
| Food Category | Use Level (ppm) |
| Breakfast cereals | <500 |
| Cheese | <250 |
| Chewing Gum | <1000 |
| Condiments & Relishes | <250 |
| Confectionery Frostings | <100 |
| Frozen Dairy | <250 |
| Fruit Ices | <250 |
| Gelatins & Puddings | <250 |
| Gravies | <250 |
| Hard Candy | <300 |
| Imitation Dairy Products | <250 |
| Instant Coffee & Tea | <250 |
| Seasonings & Flavors | <400 |
| Snack Foods | <500 |
| Soft Candy | <300 |
| Soups | <300 |
| Sugar Substitutes | <800 |
| Sweet Sauce | <400 |

In conclusion, it has been unexpectedly discovered that glucosylation of steviol glycosides beyond a certain number of glucose units effectively reduces sweetness and contributes to the modification of flavor and sweetness profiles. Therefore, while sweetness decreases with glucosylation, flavor modification increases.

The above examples of GSG application in different food and beverage system illustrate the contribution of GSG to flavor and sweetness profile modification. GSG modifies flavor by increasing intensity of some flavor and/or suppressing other flavor notes. Thus GSG is a flavor modifier and not a flavor enhancer. These examples also show the synergistic effect of GSG with caloric sweeteners (Sugar, HFCS, and Fructose) on the overall sweetness perception. The key finding of these studies is that a modest increase in sweetness perception and flavor modification occur simultaneously in food system with GSG, which cannot be decoupled for a complex product matrix.

Another key observation on GSG application is that solid food (e.g., chewing gum, snacks, breakfast cereal, etc) and semi-solid food (e.g., paste, yogurt, fruit prep) need higher concentration (ppm) of GSG compared to beverage, to deliver flavor modification, because of the mastication, residence time in the mouth and dilution effect during consumption.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the invention described in the specification. As one of ordinary skill in the art will readily appreciate from the disclosure of the present invention, the compositions, devices, processes, methods, and steps, presently existing or later to be developed that perform substantially the same function or achieve substantially the same result as the corresponding embodiments described herein may be utilized according to the present invention.

What is claimed is:

1. A taste and flavor modifying composition, comprising glucosylated steviol glycosides.

2. The taste and flavor modifying composition of claim 1, wherein the glucosylated steviol glycosides comprise more than two glucose units.

3. The taste and flavor modifying composition of claim 1, wherein the glucosylated steviol glycosides comprise more than three glucose units.

4. The taste and flavor modifying composition of claim 2, wherein at least one glucose unit occurs at position C-19 of the steviol glycoside.

5. The taste and flavor modifying composition of claim 1, further comprising dextrin.

6. A food or beverage product having an intense taste and flavor profile comprising the taste and flavor modifying composition of claim 1.

7. The food or beverage product of claim 6, selected from the group consisting of carbonated soft drink, fruit juice drink, dairy food including cheese and frozen dairy products, dairy beverages, alcoholic beverages, baked goods, processed fruit and vegetable products, gelatins/puddings, confectionery and chewing gum, soups, sauces, gravies, breakfast cereals, snacks including nuts products and table top sweetener.

8. The food or beverage product of claim 6, wherein the intense taste and flavor profile is more intense than a comparative taste and flavor profile of a comparative food or beverage product which does not include the taste and flavor modifying composition.

9. The food or beverage product of claim 6, wherein a mouthfeel of said food or beverage product is improved in relation to a mouthfeel of a comparative food or beverage product which does not include the taste and flavor modifying composition.

10. A method of increasing a taste and flavor intensity of a food or beverage product, comprising the step of adding the taste and flavor modifying composition of claim 1.

11. A method for making the taste and flavor modifying composition of claim 1, comprising the steps of:
a. Extracting steviol glycosides from leaves of a Steviol rebaudiana Bertoni plant; and
b. Transglycosylating the steviol glycosides to add glucose units to the steviol glycosides.

12. The method of claim 11, wherein transglycosylating the steviol glycosides comprises enzymatic transglycosylation using an enzyme selected from the group consisting of pullulanase, isomaltase, β-galactosidase, dextrine saccharase, and cyclodextrin glucotransferase.

13. A method of improving the organoleptic properties of a food or beverage product including a high fructose syrup, comprising the step of adding the taste and flavor modifying composition of claim 1 to said food or beverage product.

14. The method of claim 12, wherein the high fructose syrup comprises a high fructose corn syrup.

15. The method of claim 13, wherein the high fructose corn syrup comprises high fructose corn syrup 42 (HFCS 42).

16. A method of making a food or beverage product, comprising the steps of:
a. adding the taste and flavor modifying composition of claim 1; and
b. adding a reduced amount of erythritol, wherein said reduced amount of erythritol is less than an amount of erythritol in a comparative food or beverage product which does not include the taste and flavor modifying composition, and wherein the food or beverage product has a mouthfeel which is similar to a mouthfeel of the comparative food or beverage product.

17. The method of claim 16, wherein said reduced amount of erythritol is in the range of about 20% to 30% by weight less than the amount of erythritol in the comparative food or beverage product.
